(19) United States
(12) Patent Application Publication
(10) Pub. No.: US 2003/0223966 A1
(43) Pub. Date: Dec. 4, 2003

(54) Treatment for pompe disease

(76) Inventors: Thomas J. Fraites JR., Gainesville, FL (US); Mary B. Rucker, Gainesville, FL (US); Barry J. Byrne, Gainesville, FL (US)

Correspondence Address: Stanley A. Kim,Ph.D., Esq., Akerman Senterfitt, Suite 400, 222 Lakeview Avenue, West Palm Beach, FL, 33402-3188, US

(21) Appl. No.: 10/426,367

(22) Filed: Apr. 30, 2003

Related U.S. Application Data
(60) Provisional application No. 60/377,311, filed on Apr. 30, 2002.

(51) Int. Cl. A61K 48/00; C12N 15/861

(52) U.S. Cl. 424/93.2; 435/456

(57) ABSTRACT

Serotype 1 recombinant adeno-associated virus (rAAV) vectors were used to deliver functional acid alpha-glucosidase genes in vitro and in vivo to muscle cells deficient in acid alpha-glucosidase. The vector-treated cells overexpressed acid alpha-glucosidase. Vector-treated animals displayed restored enzymatic activity and muscle function. Serotype 1 rAAV vectors induced significantly greater acid alpha-glucosidase expression compared to serotype 2 rAAV vectors.

### CROSS-REFERENCE TO RELATED APPLICATIONS

The present application claims the priority of U.S. provisional patent application No. 60/377,311 filed on Apr. 30, 2002.

### STATEMENT AS TO FEDERALLY SPONSORED RESEARCH

This invention was made with U.S. government support under grant number 5P50HL059412-05 awarded by the National Institutes of Health. The U.S. government may have certain rights in the invention.

### FIELD OF THE INVENTION

The invention relates generally to the fields of molecular biology, gene therapy, and medicine. More particularly, the invention relates to a gene therapy-based treatment for Pompe disease.

### BACKGROUND OF THE INVENTION

Pompe disease, also known as glycogen storage disease type II (GSDII), is an autosomal recessive disorder caused by a deficiency of the lysosomal enzyme, acid α-glucosidase (GAA). GAA is responsible for the cleavage of α-1,4 and α-1,6 linkages in lysosomal glycogen, leading to the release of monosaccharides. A loss or absence of GAA activity leads to a massive accumulation of lysosomal and cytoplasmic glycogen in striated muscle. This accumulation results in contractile dysfunction and muscle weakness (Baudhin and Hers, Lab. Invest. 13:1139-1152, 1964 and Hirschhorn and Reuser, in “The Metabolic and Molecular Bases of Inherited Disease,” C. Scriver et al., Eds., 3389-3420, Mc-Graw Hill, New York, 2000).

Pompe disease has been classified into two types, early onset and late onset. Early onset Pompe disease is characterized by a rapidly progressing cardioskeletal myopathy that culminates in cardiorespiratory failure and death within the first two years of life (Hirschhorn and Reuser, Id.; Hers, Biochem. J. 86:11, 1963; and Reuser et al., Muscle Nerve, 3:S61-S69, 1995). Late onset Pompe disease progresses more slowly, and is characterized by muscle weakness in the trunk, lower limbs, and diaphragm. Many patients succumb to respiratory insufficiency as a result of diaphragmatic weakness (Moufarrej and Bertorini, South. Med. J. 86:560-567, 1993).

Early attempts to treat Pompe disease included a high-protein diet, β-adrenergic drugs, thyroid and steroid hormones, and bone marrow transplantation. Each of these was largely unsuccessful (Slonim et al., Neurology 33:34-38, 1983 and Watson et al., N. Engl. J. Med. 314:385, 1986). Currently, no effective treatment is widely available, although clinical trials have begun to evaluate weekly infusion of exogenously-produced, purified recombinant GAA (Van den Hout et. al., Lancet 356:397-398, 2000; Van den Hout et al., J. Inherit. Metab. Dis. 24:266-274, 2001; and Amalfitano et al., Genet. Med. 3:132-138, 2001).

### SUMMARY

The invention relates to the discovery that serotype 1 recombinant adeno-associated virus (rAAV) vectors can direct the synthesis of very high levels of GAA in cells and animals that were previously deficient in this enzyme. The expression is significantly greater than that induced using comparable serotype 2 rAAV vectors. Moreover, it is sufficiently high that clinical manifestations of GAA deficiency can be ameliorated in animal subjects.

Accordingly, the invention features a method that includes a step of administering to a cell an rAAV virion that includes both (a) a polynucleotide encoding a GAA polypeptide (e.g., a human GAA polypeptide) interposed between a first AAV inverted terminal repeat and second AAV inverted terminal repeat; and (b) an AAV serotype 1 capsid protein. The nucleotide sequence encoding GAA polypeptide can be operably linked to an expression control sequence such as a promoter (e.g., a CMV immediate early promoter).

The cell to which the virion is administered can be a mammalian cell such as a mammalian muscle cell. The cell can be derived from an animal having lower than wild-type acid alpha-glucosidase polypeptide levels (e.g., a Pompe disease patient). It can be located within a mammalian subject including a post-natal animal and a fetus.

The step of administering the rAAV virion can be performed by parenteral administration such as by injection (e.g., intramuscular (IM) injection or injection into a blood vessel). Administration of the rAAV virion can result in increased GAA polypeptide levels (e.g., greater than or equal to wild-type levels) in the treated subject. In cases where the subject exhibits clinical symptoms associated with low GAA polypeptide levels, the symptoms can be ameliorated after the step of administering the rAAV virion.

Unless otherwise defined, all technical terms used herein have the same meaning as commonly understood by one of ordinary skill in the art to which this invention belongs.

As used herein, a “nucleic acid,” “nucleic acid molecule,” or “polynucleotide” means a chain of two or more nucleotides such as RNA (ribonucleic acid) and DNA (deoxyribonucleic acid). A “purified” nucleic acid molecule is one that has been substantially separated or isolated away from other nucleic acid sequences in a cell or organism in which the nucleic acid naturally occurs (e.g., 30, 40, 50, 60, 70, 80, 90, 95, 96, 97, 98, 99, 100% free of contaminants). The term includes, e.g., a recombinant nucleic acid molecule incorporated into a vector, a plasmid, a virus, or a genome of a prokaryote or eukaryote.

As used herein, “protein” or “polypeptide” are used synonymously to mean any peptide-linked chain of amino acids, regardless of length or post-translational modification, e.g., glycosylation or phosphorylation.

When referring to a nucleic acid molecule or polypeptide, the term “native” refers to a naturally-occurring (e.g., a wild-type; “WT”) nucleic acid or polypeptide.

As used herein, the term “vector” refers to a nucleic acid molecule capable of transporting another nucleic acid to which it has been linked. Vectors capable of directing the expression of genes to which they are operatively linked are referred to herein as “expression vectors.”

A first nucleic acid sequence is “operably” linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein coding regions, in reading frame.

As used herein, the phrase “expression control sequence” refers to a nucleic acid that regulates the replication, transcription and translation of a coding sequence in a recipient cell. Examples of expression control sequences include promoter sequences, polyadenylation (pA) signals, introns, transcription termination sequences, enhancers, upstream regulatory domains, origins of replication, and internal ribosome entry sites (“IRES”). The term “promoter” is used herein to refer to a DNA regulatory sequence to which RNA polymerase binds, initiating transcription of a downstream (3′ direction) coding sequence.

By the term “pseudotyped” is meant a nucleic acid or genome derived from a first AAV serotype that is encapsidated or packaged by an AAV capsid containing at least one AAV Cap protein of a second serotype. By “AAV inverted terminal repeats”, “AAV terminal repeats, “ITRs”, and “TRs” are meant those sequences required in cis for replication and packaging of the AAV virion including any fragments or derivatives of an ITR which retain activity of a full-length or WT ITR.

As used herein, the terms “rAAV vector” and “recombinant AAV vector” refer to a recombinant nucleic acid derived from an AAV serotype, including without limitation, AAV1, AAV2, AAV3, AAV4, AAV5, AAV6, AAV7, etc. rAAV vectors can have one or more of the AAV WT genes deleted in whole or in part, preferably the rep and/or cap genes, but retain functional flanking ITR sequences. A “recombinant AAV virion” or “rAAV virion” is defined herein as an infectious, replication-defective virus composed of an AAV protein shell encapsulating a heterologous nucleotide sequence that is flanked on both sides by AAV ITRs.

By the term “rAAV1” is meant a rAAV virion having at least one AAV serotype 1 capsid protein. Similarly, by the term “rAAV2” is meant a rAAV virion having at least one AAV serotype 2 capsid protein.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is a pair of graphs showing in vitro expression and lysosomal targeting of GAA in cells from GSDII patients.

FIG. 2 is a graph showing expression of recombinant human GAA in BALB/c mice after transduction with rAAV2-hGAA.

FIG. 3 is a pair of graphs illustrating rAAV2-mGaa-mediated transduction of skeletal and cardiac muscle in Gaa mice.

FIG. 4 is a graph showing force-frequency relationships of intact soleus muscles after direct IM delivery of rAAV2-mGaa.

FIG. 5 is a graph and proton nuclear magnetic resonance (¹H-NMR) spectra illustrating rAAV1-mGaa-mediated transduction of skeletal muscle in Gaa⁻/⁻ mice.

FIG. 6 is a map of plasmid pXYZ1.

FIG. 7 is a pair of graphs showing the levels of enzyme activity measured in the quadriceps femoris (A) and soleus (B) muscles of Gaa−/− mice after delivery of rAAV1-mGaa.

FIG. 8 is a table indicating GAA enzymatic activity in other tissues after delivery of rAAV1-mGaa to the quadriceps femoris. The enzymatic activities are reported as a percentage of enzyme activities observed in these tissues in normal (WT) mice.

FIG. 9 is a pair of graphs showing comparable expression of GAA in cardiac tissue after direct injection of (B) rAAV1 and (A) rAAV2.

### DETAILED DESCRIPTION

The invention encompasses compositions and methods relating to the use of rAAV-based vectors and virions for transferring genetic material encoding GAA into a host cell or organism lacking normal GAA activity. The below described preferred embodiments illustrate adaptations of these compositions and methods. Nonetheless, from the description of these embodiments, other aspects of the invention can be made and/or practiced based on the description provided below.

Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., Gene Therapy: Principles and Applications, ed. T. Blackenstein, Springer Verlag, 1999; Gene Therapy Protocols (Methods in Molecular Medicine), ed. P. D. Robbins, Humana Press, 1997; and Retro-vectors for Human Gene Therapy, ed. C. P. Hodgson, Springer Verlag, 1996.

Nucleic Acids for Modulating GAA Expression

Transfer of a functional GAA protein into a cell or animal is accomplished using a nucleic acid that includes a polynucleotide encoding the functional GAA protein interposed between two AAV ITRs. The GAA-encoding polynucleotide sequence can take many different forms. For example, the sequence may be a native mammalian GAA nucleotide sequence such as one of the mouse or human GAA-encoding sequences deposited with Genbank as accession numbers NM_{—}008064, NM_{—}000152, X55080, X55079, M34425, and M34424. The GAA-encoding nucleotide sequence may also be a non-native coding sequence which, as a result of the redundancy or degeneracy of the genetic code, encodes the same polypeptide as does a native mammalian GAA nucleotide sequence. Other GAA-encoding nucleotide sequences within the invention are those that encode fragments, analogs, and derivatives of a native GAA protein. Such variants may be, e.g., a naturally occurring allelic variant of a native GAA-encoding nucleic acid, a homolog of a native GAA-encoding nucleic acid, or a non-naturally occurring variant of native GAA-encoding nucleic acid. These variants have a nucleotide sequence that differs from native GAA-encoding nucleic acid in one or more bases. For example, the nucleotide sequence of such variants can feature a deletion, addition, or substitution of one or more nucleotides of a native GAA-encoding nucleic acid. Nucleic acid insertions are preferably of about 1 to 10 contiguous nucleotides, and deletions are preferably of about 1 to 30 contiguous nucleotides. In most applications of the invention, the polynucleotide encoding a GAA substantially maintains the ability to convert phenylalanine to tyrosine.

The GAA-encoding nucleotide sequence can also be one that encodes a GAA fusion protein. Such a sequence can be made by ligating a first polynucleotide encoding a GAA protein fused in frame with a second polynucleotide encoding another protein (e.g., one that encodes a detectable label). Polynucleotides that encode such fusion proteins are useful for visualizing expression of the polynucleotide in a cell.

In order to facilitate long term expression, the polynucleotide encoding GAA is interposed between first and second AAV ITRs. AAV ITRs are found at both ends of a WT AAV genome, and serve as the origin and primer of DNA replication. ITRs are required in cis for AAV DNA replication as well as for rescue, or excision, from prokaryotic plasmids. The AAV ITR sequences that are contained within the nucleic acid can be derived from any AAV serotype (e.g., 1, 2, 3, 4, 5, 6 and 7) or can be derived from more than one serotype. For use in a vector, the first and second ITRs should include at least the minimum portions of a WT or engineered ITR that are necessary for packaging and replication.

In addition to the AAV ITRs and the polynucleotide encoding GAA, the nucleic acids of the invention can also include one or more expression control sequences operatively linked to the polynucleotide encoding GAA. Numerous such sequences are known. Those to be included in the nucleic acids of the invention can be selected based on their known function in other applications. Examples of expression control sequences include promoters, insulators, silencers, response elements, introns, enhancers, initiation sites, termination signals, and pA tails.

To achieve appropriate levels of GAA, any of a number of promoters suitable for use in the selected host cell may be employed. For example, constitutive promoters of different strengths can be used. Expression vectors and plasmids in accordance with the present invention may include one or more constitutive promoters, such as viral promoters or promoters from mammalian genes that are generally active in promoting transcription. Examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Examples of constitutive mammalian promoters include various housekeeping gene promoters, as exemplified by the β-actin promoter. As described in the examples below, the chicken beta-actin (CB) promoter has proven to be a particularly useful constitutive promoter for expressing GAA.

Inducible promoters and/or regulatory elements may also be contemplated for use with the nucleic acids of the invention. Examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are useful in certain embodiments of the invention. Examples of such promoters that may be used with the expression vectors of the invention include (1) creatine kinase, myogenin, alpha myosin heavy chain, human brain and natriuretic peptide, specific for muscle cells, and (2) albumin, alpha-1-antitrypsin, hepatitis B virus core protein promoters, specific for liver cells.

rAAV Vectors and Virions

The nucleic acids of the invention may be incorporated into vectors and/or virions in order to facilitate their introduction into a cell. rAAV vectors useful in the invention are recombinant nucleic acid constructs that include (1) a heterologous sequence to be expressed (e.g., a polynucleotide encoding a GAA protein) and (2) viral sequences that facilitate integration and expression of the heterologous genes. The viral sequences may include those sequences of AAV that are required in cis for replication and packaging (e.g., functional ITRs) of the DNA into a virion. In preferred applications, the heterologous gene encodes GAA, which is useful for correcting a GAA-deficiency in a cell. Such rAAV vectors may also contain marker or reporter genes. Useful rAAV vectors have one or more of the AAV WT genes deleted in whole or in part, but retain functional flanking ITR sequences. The AAV ITRs may be of any serotype (e.g., derived from serotype 2) suitable for a particular application. Methods for using rAAV vectors are discussed, for example, in Tal, J., J. Biomed. Sci. 7:279-291, 2000 and Monahan and Samulski, Gene delivery 7:24-30, 2000.

The nucleic acids and vectors of the invention may be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described. See, e.g., U.S. Pat. Nos. 5,173,414, 5,139,941, 5,863,541, and 5,869,305, 6,057,152, 6,376,237; Rabinowitz et al., J. Virol. 76:791-801, 2002; and Bowles et al., J. Virol. 77:423-432, 2003.

rAAV virions useful in the invention include those derived from a number of AAV serotypes including 1, 2, 3, 4, 5, 6, and 7. For targeting muscle cells, rAAV virions that include at least one serotype 1 capsid protein are preferred as the experiments reported herein show they induce significantly higher cellular expression of GAA than do rAAV virions having only serotype 2 capsids. Also preferred are rAAV virions that include at least one serotype 6 capsid protein as serotype 6 capsid proteins are structurally similar to serotype 1 capsid proteins, and thus are expected to also result in high expression of GAA in muscle cells. Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

Also useful in the invention are pseudotyped rAAV. Pseudotyped vectors of the invention include AAV vectors of a given serotype (e.g., AAV2) pseudotyped with a capsid gene derived from a serotype other than the given serotype (e.g., AAV1, AAV3, AAV4, AAV5, AAV6 or AAV7 capsids). For example, a representative pseudotyped vector of the invention is an AAV2 vector encoding GAA pseudotyped with a capsid gene derived from AAV serotype 1, as serotype 1 has shown enhanced infectivity of muscle cells compared to other serotypes. Techniques involving the construction and use of pseudotyped rAAV virions are known in the art and are described in Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet. 10:3075-3081, 2001.

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635-45, 2000. Other rAAV virions that can be used in methods of the invention include those capsid hybrids that are generated by molecular breeding of viruses as well as by exon shuffling. See Soong et al., Nat. Genet. 25:436-439, 2000; and Kolman and Stemmer Nat. Biotechnol. 19:423-428, 2001.

Modulating GAA Levels in a Cell

The nucleic acids, vectors, and virions described above can be used to modulate levels of GAA in a cell. The method includes the step of administering to the cell a composition including a nucleic acid that includes a polynucleotide encoding GAA interposed between two AAV ITRs. The cell can be from any animal into which a nucleic acid of the invention can be administered. Mammalian cells (e.g., human beings, dogs, cats, pigs, sheep, mice, rats, rabbits, cattle, goats, etc.) from a subject with GAA deficiency are typical target cells for use in the invention.

Increasing GAA Activity in a Subject

The nucleic acids, vectors, and virions described above can be used to modulate levels of functional GAA in an animal subject. The method includes the step of providing an animal subject and administering to the animal subject a composition including a nucleic acid that includes a polynucleotide encoding GAA interposed between two AAV ITRs. The subject can be any animal into which a nucleic acid of the invention can be administered. For example, mammals (e.g., human beings, dogs, cats, pigs, sheep, mice, rats, rabbits, cattle, goats, etc.) are suitable subjects. The methods and compositions of the invention are particularly applicable to GAA-deficient animal subjects.

The compositions described above may be administered to animals including human beings in any suitable formulation by any suitable method. For example, rAAV virions (i.e., particles) may be directly introduced into an animal, including by intravenous (IV) injection, intraperitoneal (IP) injection, or in situ injection into target tissue (e.g., muscle). For example, a conventional syringe and needle can be used to inject an rAAV virion suspension into an animal. Depending on the desired route of administration, injection can be in situ (i.e., to a particular tissue or location on a tissue), IM, IV, IP, or by another parenteral route. Parenteral administration of virions by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the rAAV virions may be in powder form (e.g., lyophilized) for constitution with a suitable vehicle, for example, sterile pyrogen-free water, before use.

To facilitate delivery of the rAAV virions to an animal, the virions of the invention can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly preferred for delivery of virions to human subjects. Methods for making such formulations are well known and can be found in, for example, Remington's Pharmaceutical Sciences.

In addition to the formulations described previously, the virions can also be formulated as a depot preparation. Such long acting formulations may be administered by implantation (for example subcutaneously or intramuscularly) or by IM injection. Thus, for example, the virions may be formulated with suitable polymeric or hydrophobic materials (for example as an emulsion in an acceptable oil) or ion exchange resins, or as sparingly soluble derivatives.

Similarly, rAAV vectors may be administered to an animal subject using a variety of methods. rAAV vectors may be directly introduced into an animal by peritoneal administration (e.g., IP injection, oral administration), as well as parenteral administration (e.g., IV injection, IM injection, and in situ injection into target tissue). Methods and formulations for parenteral administration described above for rAAV virions may be used to administer rAAV vectors.

Ex vivo delivery of cells transduced with rAAV virions is also provided for within the invention. Ex vivo gene delivery may be used to transplant rAAV-transduced host cells back into the host. Similarly, ex vivo stem cell (e.g., mesenchymal stem cell) therapy may be used to transplant rAAV vector-transduced host cells back into the host. A suitable ex vivo protocol may include several steps. A segment of target tissue (e.g., muscle, liver tissue) may be harvested from the host and rAAV virions may be used to transduce a GAA-encoding nucleic acid into the host's cells. These genetically modified cells may then be transplanted back into the host. Several approaches may be used for the reintroduction of cells into the host, including intravenous injection, intraperitoneal injection, or in situ injection into target tissue. Microencapsulation of cells transduced or infected with rAAV modified ex vivo is another technique that may be used within the invention. Autologous and allogeneic cell transplantation may be used according to the invention.

Effective Doses

The compositions described above are preferably administered to a mammal in an effective amount, that is, an amount capable of producing a desirable result in a treated subject (e.g., increasing WT GAA activity in the subject). Such a therapeutically effective amount can be determined as described below.

Toxicity and therapeutic efficacy of the compositions utilized in methods of the invention can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one animal depends on many factors, including the subject's size, body surface area, age, the particular composition to be administered, time and route of administration, general health, and other drugs being administered concurrently. It is expected that an appropriate dosage for intravenous administration of particles would be in the range of about 10¹²-10¹⁵ particles. For a 70 kg human a 1-10 ml (e.g., 5 ml) injection of 10¹²-10¹⁵ particles is presently believed to be an appropriate dose.

### EXAMPLES

### Example 1

Materials and Methods

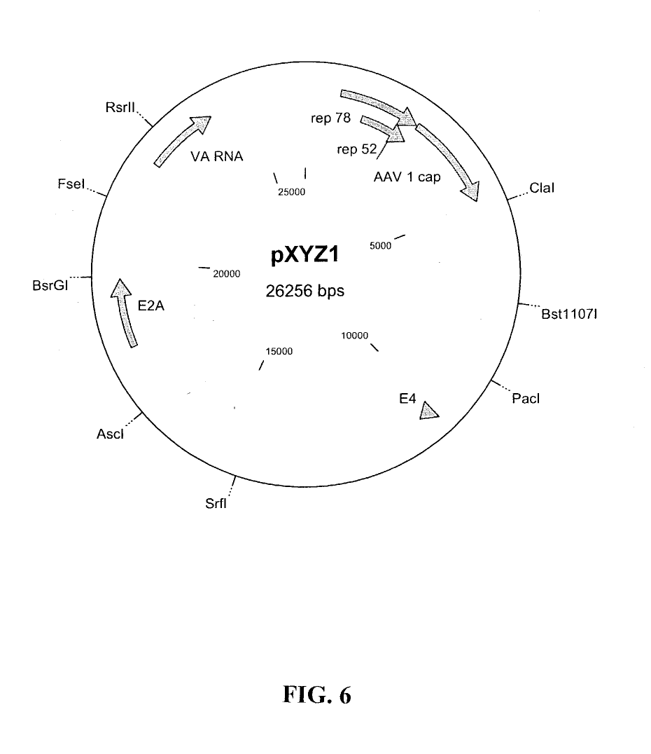

rAAV1 vectors were created using a cross-packaging method similar to the ones described in Xiao et al., J Virol. May 1999;73(5):3994-4003 and Rabinowitz et al., J Virol. January 2002;76(2):791-801. The human and mouse cDNAs encoding GAA were cloned into an rAAV2 vector plasmid (containing the AAV2 ITRs) as described previously (U.S. Pat. Nos. 5,139,941 and 5,962,313). To generate serotype 1 vectors, a new plasmid (pXYZ1) was cloned, encoding adenovirus helper genes, replication genes from AAV2, and capsid genes from AAV1. A map of the plasmid is shown in FIG. 6. The packaging protocol used to generate AAV1 vectors was identical to the method described in U.S. Pat. No. 6,146,874.

Molecular cloning of rAAV vectors carrying the human GAA and murine Gaa genes. The human GAA and murine Gaa cDNAs (hGAA and mGaa), respectively, were constructed as described previously by Pauly et al., (Hum. Gene Ther. 12:527-538, 2001). The full-length cDNAs were placed under the transcriptional control of the CMV immediate early promoter in the mammalian expression plasmid pCI (Clontech, Palo Alto, Calif.), yielding pCI-hGAA and pCI-mGaa. The expression cassettes were then cloned into p43.2, a plasmid containing both of the AAV serotype 2 ITRs. The human vector plasmid, p43.2-hGAA, was generated via EcoRI-XbaI, and p43.2-mGaa was similarly cloned via SpeI-MunI. A control recombinant AAV vector plasmid (pAAV-βgal) carrying the gene for Escherichia coli β-galactosidase under the transcriptional control of the CMV promoter has been described previously by Kessler et al., (PNAS 93:14082-14087, 1996).

To confirm the enzymatic activity of recombinant GAA produced from p43.2-hGAA and p43.2-mGaa, rAAV vector plasmids were transfected into COS-1 cells, and GAA activity was measured 72 h after transfection, as described below. An eight-to ten-fold increase in activity was observed after transfection with p43.2-hGAA or p43.2-mGaa, compared to untransfected cells or cells transfected with pAAV-βgal. The DNA sequences for the two rAAV GAA plasmids were confirmed using an automated sequencing protocol. Infectious rAAV2-hGAA, rAAV2-mGaa, and rAAV2-βgal vectors were packaged and titered as described previously by Grimm et al., (Hum. Gene Ther. 9:2745-2760, 1998), Xiao et al., (J. Virol. 72:2224-2232, 1998) and Zolotukhin et al., (Gene Ther. 6:973-985, 1999). The packaging protocol yields AAV particles that have a ratio of DNA-containing to infectious particle ratio of <100. Sodium dodecyl sulfate polyacrylamide gel electrophoresis (SDS/PAGE) and silver stain, infectious center assay, particle count, and electron microscopy were used to fully characterize high-titer vector stocks (up to 1×10¹¹ infectious units (i.u.)/mL). Similar techniques were used to produce and isolate rAAV1-mGaa vectors.

Cell lines and in vitro and in vivo viral transduction. Cultured cells were maintained in 5% CO₂ at 37° C. GAA-deficient fibroblasts isolated from an infant with GSDII (GM04912 cells) were obtained from the NIGMS Mutant Cell Repository (Camden, N.J.). Normal human skeletal muscle cells were obtained from Clonetics Corporation (Walkersville, Md.). GM04912 cells were cultured in 24-well plates at a density of 1×10⁵ in growth medium (GM; 20% [vol/vol] fetal calf serum (FCS) in DMEM). In vitro transduction with rAAV was performed in Opti-MEM, and after viral adsorption, cells were cultured in 2% FCS in DMEM. Normal and deficient human myoblasts were seeded in 24-well plates at a density of 2×10⁴ cells/cm² and cultured to confluence in GM. Once the cells reached confluence, differentiation medium (DM; 2% [vol/vol] horse serum in DMEM) was substituted to induce myoblast fusion and myotube formation. After 14 days of incubation in DM, myotubes were transduced with purified rAAV vectors in Opti-MEM. DM was reintroduced after viral adsorption. All media and sera were purchased from Life Technologies (Gaithersburg, Md.).

For animal experiments, the techniques and protocols used were identical to those described in U.S. Pat. Nos. 5,858,351, 6,211,163, and 6,335,011. Knockout (Gaa³¹ /⁻) mice (a null animal model of glycogen storage disorder type II) were treated with rAAV1-mGaa (encoding the mouse Gaa gene) and then analyzed for restoration of enzymatic activity and concomitant glycogen clearance. This resulted in functional enzymatic replacement and glycogen clearance in the treated mice.

Delivery of recombinant viral vectors to mouse skeletal muscle has been previously described by Kessler et al., (PNAS 93:14082-14087, 1996). BALB/c mice were anesthetized with inhaled methoxyflurane, and 1×10⁹ i.u. of rAAV2-hGAA or rAAV2-βgal were injected into the TA muscle after minimal exposure of the muscle via a single incision. For IMrAAV2-mGaa experiments, rAAV2-mGaa (1×10⁹ i.u.) was injected into the quadriceps muscle of Gaa⁻/⁻ mice (Raben et al., J. Biol. Chem. 273:19086-19092, 1998) using minimal exposure; mice were then sutured as described before. Control mice of the same genetic background (C57BL6/129SvJ) were injected with identical volumes of sterile saline.

To facilitate direct injection into cardiac muscle, adult Gaa⁻/⁻ and control mice were anesthetized with an IP injection of a ketamine/xylazine (100 mg/kg ketamine; 15 mg/kg xylazine) cocktail. Animals were placed in a supine position in a sterile surgical field. The trachea was exposed and a 22G catheter was introduced to facilitate ventilation using an SAR-830AP rodent ventilator (CWE, Ardmore, Pa.). The animal was ventilated at 110 breaths/min with a tidal volume of 0.2 cc/min. A left thoracotomy was performed, and the ribs were retracted to give full visualization of the left ventricle. Injections of 10 to 50 μL were carried out with a 29-gauge insulin syringe. The ribs and skin were closed, and the animal was weaned from the ventilator. All animals were monitored overnight for pain or distress and for a week or more for infection or other complications.

Assays of GAA activity and glycogen concentration. Enzymatic activity assays for GAA were performed as described previously by Pauly et al., (Gene Ther. 5: 473-480, 1998). Transduced tissue culture cells were harvested and lysed in a commercial lysis buffer (Analytic Luminescence Lab). Alternatively, harvested muscle tissues were homogenized in water, then subjected to three freeze-thaw cycles. Lysates were centrifuged, and clarified supernatants were assayed for GAA activity by measuring the cleavage of the synthetic substrate 4-methylumbelliferyl-α-D-glucoside (Sigma M9766, Sigma-Aldrich, St. Louis, Mo.) after incubation for 1 h at 37° C. Successful cleavage yielded a fluorescent product that emits at 448 nm, as measured with a TKO100 fluorometer. Protein concentration was measured using a standard bicinchoninic acid method (Bio-Rad, Hercules, Calif.), with bovine serum albumin as a standard. Data are represented as nanomoles of substrate cleaved in one hour per milligram of total protein in the lysate (nmol/hr/mg). Glycogen concentration was assessed by measuring the amount of glucose released from tissue homogenates after treatment with amyloglucosidase as described previously by Amalfitano et al., (PNAS96:8861-8866, 1999) and Kikuchi, T. et al., (J. Clin. Invest. 101:827-833, 1998).

Immunocytochemistry. For immunofluorescence microscopy, cells on coverslips were fixed with 50% methanol/50% acetone (vol/vol) at −20° C. for 15 min. Samples were blocked with 50% FBS/50% phoshpate-buffered saline (PBS) (vol/vol) for 1 h at room temperature, then incubated for 1 h at 25° C. with a previously described rabbit-derived anti-human GAA antiserum (Pauly et al., Gene Ther. 5: 473-480, 1998), diluted 1:1000 in PBS with 50% FCS and 0.01% NaN₃. Cells were washed in PBS three times and incubated for 1 h at 25° C. with fluorescein isothiocyanate-conjugated goat anti-rabbit antibody. The slips were again washed three times, mounted with an aqueous/dry-mounting medium (Biomeda, Foster City, Calif.) and examined with fluorescence microscopy. For localization of human GAA in the lysosomal compartment, transduced cells were fixed and probed simultaneously with a mouse monoclonal antibody recognizing human lysosome-associated membrane protein 1 (LAMP-1) and rabbit anti-human acid α-glucosidase antiserum. Cells were incubated with tetramethyl rhodamine-conjugated goat anti-mouse IgG and fluorescein-conjugated goat anti-rabbit IgG.

Perchloric acid extraction and ¹H-NMR spectroscopy. Mice were fasted overnight to lower background glycogen to minimal levels. Upon sacrifice, samples were prepared by rapid freezing in liquid nitrogen and pulverization into a fine powder. Liquid nitrogen was evaporated and the powder was transferred to a 15 mL polypropylene tube containing 3 mL 7% (vol/vol) perchloric acid in 50 mM NaH₂PO₄. The sample was vortexed repeatedly and centrifuged at 4° C. and 4,000 rpm for 15 minutes. The supernatant was transferred to a new tube and neutralized to pH 7.0 with 5M potassium hydroxide, leading to precipitate formation. The precipitate was removed by centrifugation; the supernatant was transferred to a new tube, and paramagnetic metals and excess salts were removed by incubation with pre-washed Chelex beads at a 1:8 ratio for 20 minutes at 4° C. The mixture was filtered through a 0.22 μm filter and lyophilized overnight. Samples were resuspended in D₂O for spectroscopy.

¹H-NMR measurements were performed using a Bruker Avance 500 spectrometer with an 11.75 T Magnex. Spectra were collected under unsaturated conditions at 25° C. and pH 7.0 (TR=5s, sweep width=6.666 KHz, pulse width=5.5 μsec, number of averages=256, number of points=40K). Integrated areas and chemical shifts were referenced to the total creatine peak (3.0 ppm) for each sample.

Assessment of skeletal muscle function. Direct IM injections of rAAV2-mGaa (2×10⁹ i.u.) or lactated Ringer's were performed in the soleus muscle of Gaa⁻/⁻ mice. After six weeks, the mechanical function of the muscles was assessed. Gaa⁻/⁻ and C57BL6/129SvJ controls were anesthetized via IP injection of ketamine/xylazine. After reaching a surgical plane of anesthesia, the soleus muscles were surgically excised and placed in a cooled dissecting chamber containing Krebs-Henseleit solution, equilibrated with a 95% O₂/5% CO₂ gas mixture. The intact muscles were then vertically suspended between two lightweight Plexiglas clamps connected to force transducers (Model FT03, Grass Instruments, West Warwick, RI) in a water-jacketed tissue bath containing Krebs-Henseleit solution equilibrated with a 95% O₂/5% CO₂ gas (bath˜37±0.5° C., pH˜7.4±0.05, osmolality˜290 mOsmol). Transducer outputs were amplified and differentiated by operational amplifiers and undergo A/D conversion for analysis using a computer based data acquisition system (Polyview, Grass Instruments).

In vitro contractile measurements begin with empirical determination of the muscle's optimal length (Lₒ) for isometric tetanic tension development. The muscle is field-stimulated using a stimulator (Model S48, Grass Instruments) along its entire length with platinum electrodes. Muscle length is progressively increased until maximal isometric twitch tension is obtained. Once the highest twitch force is achieved, all contractile properties are measured isometrically at Lₒ. The force-frequency relationship is examined using previously described methods (Dodd et al., Med. Sci. Sports Exerc. 28:669-676, 1996).

### Example 2

Results

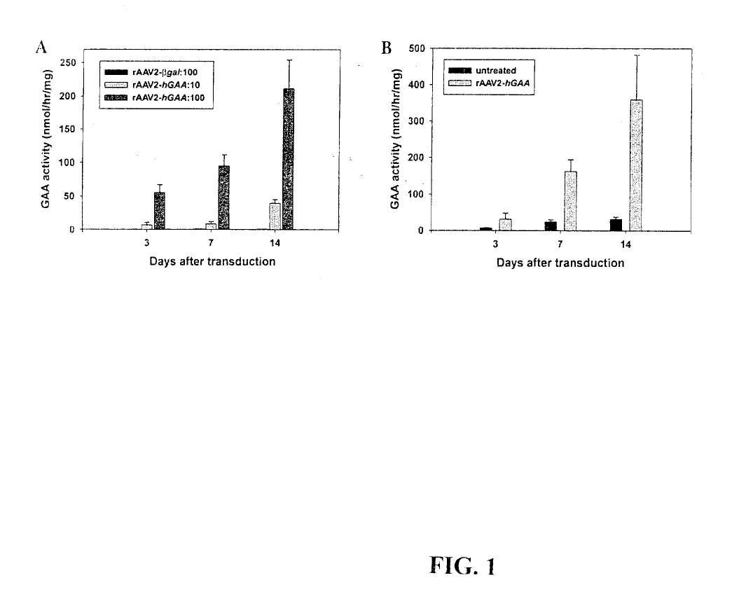

Human GAA is expressed and is enzymatically active in GSDII cells after in vitro transduction with rAAV2-hGAA. The expression of recombinant human GAA in deficient fibroblasts and myotubes from patients with GSDII was examined. Fibroblasts of GSDII patients were grown in 24-well plates and transduced with rAAV2-hGAA or rAAV2-βgal in 2% fetal bovine serum/DMEM and harvested at 3, 7, or 14 days after introduction of rAAV vectors. GAA activity was assayed as described. rAAV2-βgal:100, control cells transduced rAAV2-βgal at a multiplicity of infection (MOI) of 100; rAAV2-hGAA:10 and rAAV2-hGAA:100, cells transduced with a rAAV2-hGAA at an MOI of 10 and 100, respectively (FIG. 1A). Bar graph represents mean<SEM of GAA activities from independent triplicate cultures. Deficient fibroblasts have no GAA activity, whereas deficient myotubes retain 50 to 80% of the GAA activity of normal human myotubes. Fourteen days after rAAV2-hGAA transduction, GAA activity in deficient fibroblasts reached 30% of normal with an MOI of 10, whereas GAA activities of 150% normal were observed at an MOI of 100 (FIG. 1A). In deficient myotubes transduced with rAAV2-hGAA at an MOI of 10, a 10-fold increase (360.0<122.9 v. 32.0<5.3 nmol/hr/mg) in enzymatic activity was observed 2 weeks after transduction (FIG. 1B). GSDII myoblasts were seeded in 24-well plates at a density of 2×10⁴ cells/cm² and cultured to confluence in growth medium; then harvested at 3, 7, or 14 days after infection with rAAV2-hGAA. GAA activities were assayed as described. Untransduced, control cells with no rAAV vector; rAAV2-hGAA, cells infected with rAAV2-hGAA at an MOI of 10. The bar graph of FIG. 1B represents mean<SEM of GAA activities from independent triplicate cultures. These data indicate that rAAV2-hGAA is capable of restoring GAA activity in deficient cells in vitro in a dose-dependent manner.

To confirm that recombinant human GAA was being properly expressed and localized intracellularly, vector-derived human GAA protein was probed for in transduced, deficient cells. Immunofluorescent staining of human deficient fibroblasts transduced with rAAV2-hGAA showed that the protein is correctly targeted to the cytoplasm, with a lysosomal distribution pattern. To confirm the lysosomal targeting of GAA, co-localization of GAA and LAMP-1, a specific marker for mature lysosomes, was tested. Fibroblasts from a GSDII patient were incubated with an anti-hGAA antibody and a FITC-conjugated secondary antibody 8 days after infection with rAAV2-hGAA. The same cells were also incubated with an anti-LAMP-1 antibody and a rhodamine-conjugated secondary antibody. A digitally-merged FITC/rhodamine image showed co-localization of the two signals in yellow, confirming that recombinant human GAA is sorted to the lysosomal compartment. Positive staining for GAA was coincident with the LAMP-1 staining indicating that GAA protein expressed from rAAV2-hGAA is indeed transported to lysosomes.

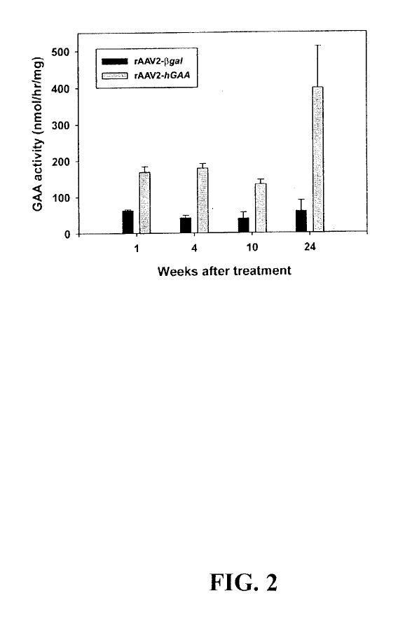

In vivo delivery of rAAV2 vectors results in stable, long-term expression of human or mouse GAA in mouse muscle. To examine the efficiency and stability of rAAV2-mediated expression of GAA, the vectors were tested in vivo by injecting 1×10⁹ i.u. of rAAV2-hGAA into the TA muscles of adult BALB/c mice. Muscle tissues were isolated at 1 week, 4 weeks, 10 weeks, and 6 months after treatment (FIG. 2), and assayed for GAA activity. The bar graph represents mean<SEM GAA activity in five animals (weeks 1 and 4) or four animals (weeks 10 and 24). The results showed that GAA enzymatic activity was increased over 150% in the TA muscles at 1 week (168.1<16.0 nmol/hr/mg treated v. 62.0<3.1 control), and this level of activity was maintained or increased over 6 months, with the highest activities observed at the latest timepoint (397.9<113.3 nmol/hr/mg). The control group, which was injected with rAAV2-βgal, showed no change in GAA enzymatic activity over the same period. These data demonstrate that the rAAV2 is capable of expressing GAA efficiently, and that the expression is stable for up to 6 months after a single IM injection.

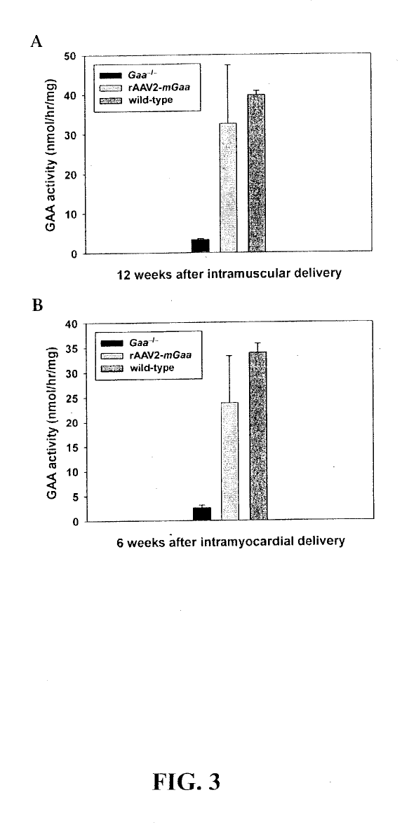

To provide further assurance that the observed enzymatic activities were not due to increased basal production in the BALB/c strain, GAA knockout mice (Gaa⁻/⁻) were treated with rAAV2-mGaa. These mice have little or no residual GAA activity and have been shown to recapitulate many of the pathologic manifestations observed in human GSDII patients (Raben et al., J. Biol. Chem. 273:19086-19092, 1998). Twelve weeks after IM delivery of 1×10⁹ i.u. of rAAV2-mGaa, normal levels of GAA enzyme activity were observed in the knockout mice (32.6±14.7 nmol/hr/mg), as compared to C57BL6/129SvJ control mice (39.7±1.0 nmol/hr/mg) (FIG. 3A). FIG. 3A shows results from adult Gaa⁻/⁻ mice treated with 1×10⁹ i.u. of rAAV2-mGaa in the quadriceps muscle. C57BL6/129SvJ controls and untreated Gaa⁻/⁻ mice were sham-injected with sterile saline. The bar graph of FIG. 3A represents mean<SEM GAA activity for five mice in each group. Similar results were obtained after intramyocardial injections (1×10⁹ i.u. rAAV2-mGaa) in Gaa⁻/⁻ mice (FIG. 3B). After intubation and a left thoracotomy, 1×10⁹ i.u. of rAAV2-mGaa⁻/⁻ were directly injected into the left ventricular free wall of Gaa knockout mice. Untreated Gaa mice were sham-injected with sterile saline. Muscle tissues were isolated 6 weeks after treatment, assayed for GAA activity, and compared to untreated age-matched C57BL6/129SvJ (WT) mice. The bar graph of FIG. 3B represents mean<SEM GAA activity for four rAAV2-mGaa-treated mice and five mice in each of the control groups. These results demonstrate that recombinant murine GAA expression can be directed by rAAV2-mGaa in both skeletal and cardiac muscle in Gaa⁻/⁻ mice.

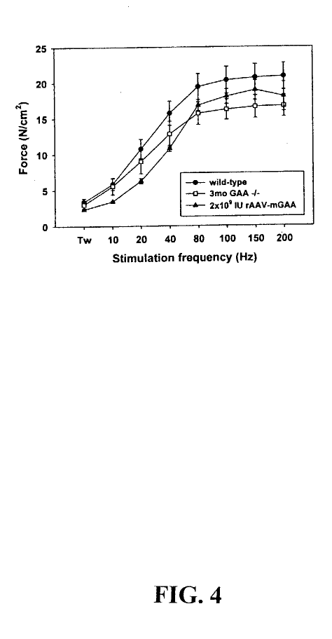

Direct IM delivery of rAAV2-mGaa preserves skeletal muscle contractile force in knockout mice. The contractile properties of soleus muscles of knockout and WT hybrid mice were analyzed using isometric force-frequency relationships as an index of contractile function. Muscles were isolated 6 weeks after treatment, tested for isometric force generation, and compared to untreated C57BL6/129SvJ (WT) (n=6) and Gaa⁻/⁻ mice (n=5), respectively. Gaa⁻/⁻ mice exhibit an age-dependent impairment of skeletal muscle function (FIG. 4, open squares), as evidenced by their decreased maximal tetanic force (16.71<1.52 N/cm²) at higher stimulation frequencies compared to the matched control strain (20.86<1.88 N/cm²; filled circles). This impairment is observed as early as three months of age (FIG. 4) and progressively worsens over the lifespan of the animal.

To test the effect of restoration of GAA activity on contractile dysfunction in Gaa⁻/⁻ mice, 2×10⁹ i.u. of rAAV2-mGaa were injected directly into the soleus muscles of six-week-olds. Isometric force generation was tested six weeks later, at three months of age (FIG. 4, filled triangles). At the maximal stimulation frequency (200 Hz), treated Gaa⁻/⁻ mice had intermediate contractile force (18.03<2.05 N/cm²) relative to untreated Gaa⁻/⁻ and WT controls. Similar relationships in isometric tension were observed between WT, treated, and untreated Gaa⁻/⁻ mice from 80 to 150 Hz, indicating some amelioration of the muscle function deficit over a range of physiologically-relevant forces.

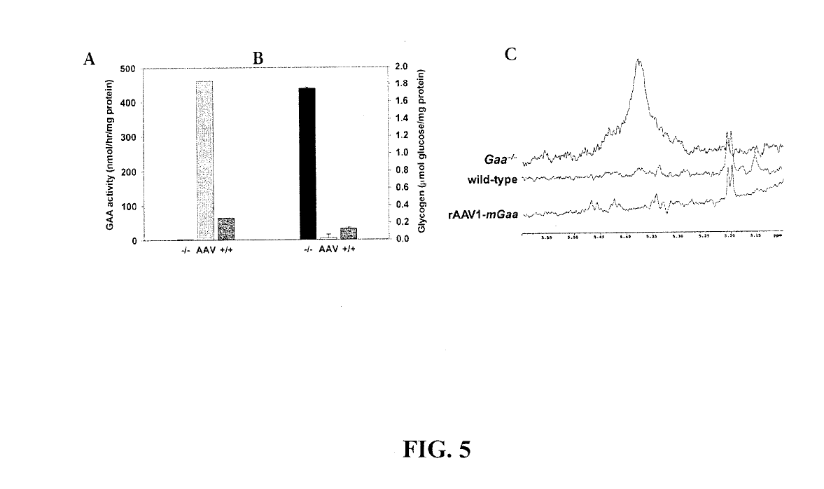

Treatment of Gaa⁻/⁻ mice with rAAV1-mGaa leads to rapid overexpression of mouse GAA and glycogen clearance. Since rAAV2-mediated gene replacement led to WT levels of GAA enzymatic activity, the ability of rAAV1 to restore GAA activity was also examined. 5×10¹⁰ total particles (as assessed by dot-blot analysis) of rAAV1-mGaa were injected directly into the TA muscles of two-month-old Gaa⁻/⁻ mice (n=4), and the mice were sacrificed two weeks later. TA muscles were harvested, pooled, and homogenized. FIG. 5A shows the results from an experiment in which 5×10¹⁰ particles of rAAV1-mGaa were directly delivered to the TA muscles of two-month-old Gaa⁻/⁻ mice (n=4). Muscles were harvested, pooled, and homogenized 2 weeks after treatment and compared to untreated C57BL6/129SvJ (WT) and Gaa⁻/⁻ mice, respectively. FIG. 5B shows in vitro glycogen content determination for the same muscle homogenates. FIG. 5C shows stacked ¹H-NMR spectra from the same homogenates after perchloric acid extraction. Glycogen peaks are observed at 5.4 ppm. GAA activities (FIG. 5A) in treated Gaa⁻/⁻ tissues (461.5 nmol/hr/mg protein) were nearly eight times WT (65 nmol/hr/mg protein). Glycogen contents of TA muscles from untreated and treated Gaa⁻/⁻ mice were 1.756 and 0.0219<mol glucose/mg protein, respectively, compared to 0.128<mol glucose/mg protein for WT mice. ¹H-NMR spectra of perchloric acid extracts from the same treated and untreated tissues showed a pronounced glycogen peak for Gaa⁻/⁻ mice and complete amelioration of glycogen accumulation in rAAV1-mGaa treated mice. Taken together, these findings indicate a dramatic reversal of glycogen accumulation after transduction with rAAV1-mGaa.

### Example 3

Correction of Glycogen Storage Disease Using rAAV1

TA muscles were directly injected with 10¹⁰ particles of rAAV1-CMV-mGAA. Injections were conducted as described in Example 1, with the sole exceptions being the muscles that were injected (quadriceps and soleus vs. TA). Two weeks after gene delivery, TA tissues were harvested and assessed for enzyme activity and glycogen content. GAA activities observed were seven-fold over WT, with complete reversal of glycogen accumulation as measured in vitro and via ¹H-NMR. The enzymatic activity assays were conducted as described in Example 1.

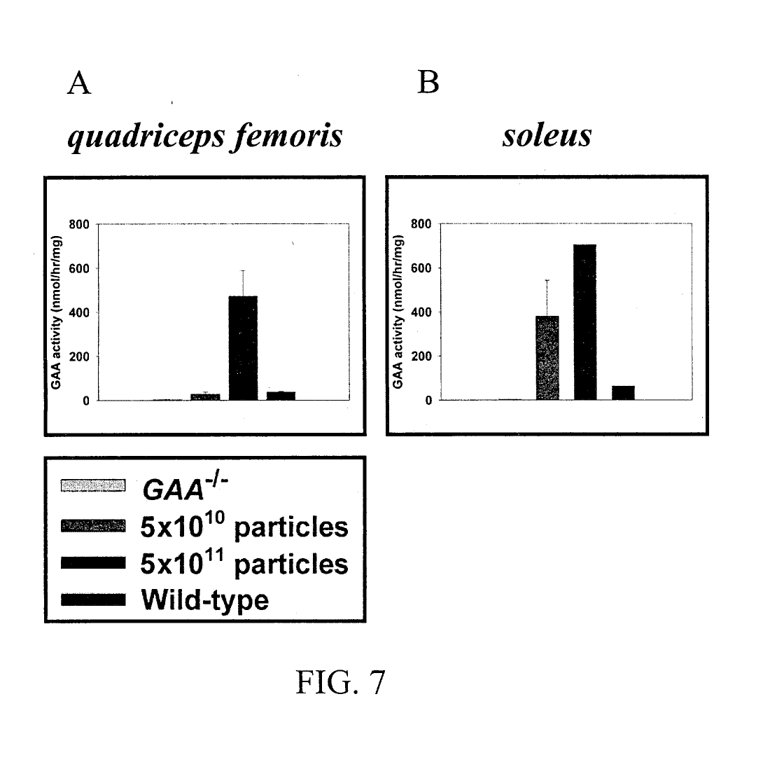

In order to assess contractile changes with restored enzyme activity, the soleus muscles of 3-month-old Gaa⁻/⁻ mice were injected with either 10¹⁰ or 5×10¹⁰ particles of rAAV1-CMVmGAA. After minimal exposure, virus was directly injected into the soleus. Animals were sacrificed 6 weeks after treatment, and isometric in vitro force-frequency relationships were measured. After functional testing, tissues were assayed for enzyme activity. The results are shown in FIG. 7B. rAAV1-CMVmGAA was able to direct enzyme synthesis in skeletal muscle (quadriceps femoris), leading to GAA activities of 381.14 and 704.89 nmol/hr/mg (low and high doses, respectively), compared to GAA⁻/⁻ and WT mice (FIG. 7A), which have activities of 2.94 and 65.01 nmol/hr/mg, respectively.

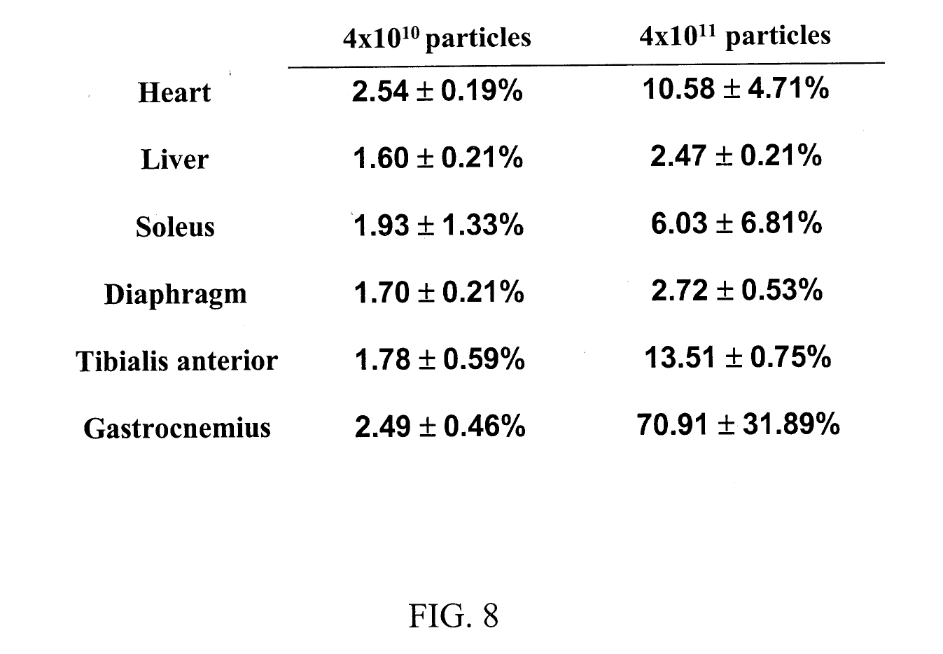

The table of FIG. 8 indicates the amount of GAA enzymatic activity measured in other tissues after delivery of rAAV1-mGaa to the quadriceps femoris. The enzymatic activities are reported as a percentage of enzyme activities observed in these tissues in normal (WT) mice. The data show that intra-cellular transfer of GAA has been achieved using rAAV delivered to the skeletal muscle. These results demonstrate the utility of rAAV1-derived lysosomal enzyme GAA in the treatment of Pompe disease.

### Example 4

Increasing Levels of Cardiac GAA Activity

To achieve higher levels of cardiac GAA activity, the ability of rAAV1vectors to direct GAA over-expression in Gaa⁻/⁻ mouse skeletal and cardiac muscle was examined.

Methods: Gaa⁻/⁻ mice were treated either by intramyocardial delivery of 10¹⁰ particles of rAAV1 carrying the human GAA cDNA under the transcriptional control of the CMV promoter (rAAV1-CMV-hGAA). Control animals were injected with similar doses of rAAV1-CMV-lacZ. Four weeks after vector delivery, mice were sacrificed and the hearts were harvested for GAA activity assay.

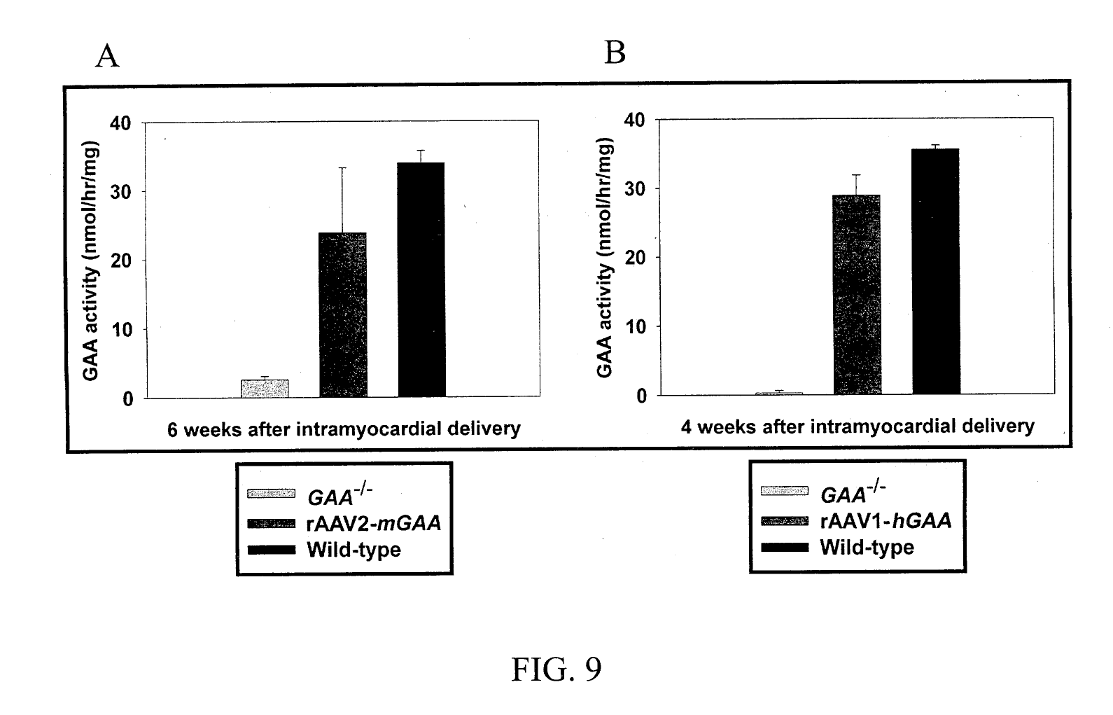

Results: The results of direct injection of rAAV1 (FIG. 9B) and rAAV2 (FIG. 9A) in cardiac tissue are shown in FIG. 9. rAAV1-CMV-hGAA was able to direct enzyme synthesis after delivery to cardiac muscle. Animal injections were conducted as described in Example 1, with the sole exception being the virus that was injected (rAAV1 vs. rAAV2). In Gaa⁻/⁻ mice treated by IM (quadriceps) injection, average cardiac GAA activities of 3.63 nmol/hr/mg were observed, compared to 0.32 and 35.5 nmol/hr/mg (lacZ vector controls and WT mice, respectively). Likewise, mice treated via direct intramyocardial delivery achieved GAA activities of 28.9 nmol/hr/mg. Enzymatic activity assays were conducted as described in Example 1.

### Example 5

Correction of GSDII in Mice After in Utero Delivery of rAAV

Gaa⁻/⁻ mice were treated with a rAAV-based gene therapy that prevented glycogen accumulation and maintained normal muscle function. In utero delivery of rAAV was used to introduce the human GAA cDNA into Gaa⁻/⁻ mice at an early stage in development and supply active GAA protein before glycogen began to accumulate, thereby preventing long-term irreversible damage to striated muscle.

Materials and Methods

Construction and preparation of viral vectors: The plasmid pCI-GAA containing the human acid α-glucosidase cDNA minus the 5′ UTR under the transcriptional control of the CMV immediate early promoter, was constructed as previously described (Pauly et al., Gene Ther. 5:473-480, 1998 and Fraites et al., Mol. Ther. 5:1-8, 2002). The plasmid p43.2-hGAA3.1 was created by cloning the CMV-hGAA expression cassette (from pCI-GAA) into p43.2, between two AAV serotype 2 ITRs. The 3′ untranslated region (UTR) of the hGAA cDNA was removed to decrease the size of the expression cassette within the ITRs. The 3′ end of the hGAA cDNA, minus the 3′ UTR, was amplified from p43.2-hGAA3.1 using a 5′ primer synthesized from bp 2285-2300 of the hGAA coding sequence and a 3′ primer containing bp 2848-2856 of the hGAA coding sequence as well as a BclI site and XbaI site. The new 3′ end of the hGAA cDNA was amplified through 35 cycles of denaturation at 95° C. for 1 minute, annealing at 60° C. for 1 minute, and elongation at 72° C. for 2 minutes using a RoboCycler® Gradient 96 thermocycler (Stratagene, La Jolla, Calif.). The plasmid, TopoII-hGAA3′ end, was created by cloning the 594 base pair PCR product into pCR-TopoII (Invitrogen Life Technologies, Carlsbad, Calif.). The 5′ portion of hGAA from p43.2-hGAA3.1 was isolated via NheI-EcoNI digestion and ligated into TopoII-hGAA3′ end after SpeI-EcoNI digestion. The human cDNA minus the 3′ UTR was cloned into p43.2 (rAAV2 expression plasmid) via like XbaI/KpnI sites to create p43.2-hGAA2.8.

An rAAV vector, pTR-CBA-hGAA3.1, containing the hGAA coding region and 3′ UTR under transcriptional control of the chicken β-actin promoter plus the CMV enhancer (CBA) was generated by replacing the CMV promoter of p43.2-hGAA3.1 with the CBA promoter found in the rAAV2 vector, UF 12. The CBA promoter fragment was released by a BglII/SpeI digest of UF12 and cloned in place of the CMV promoter after p43.2-hGAA3.1 was digested with BglII/NheI. A similar construct, pTR-CBA-hGAA2.8, containing only the hGAA coding region, was constructed by digesting both p43.2-hGAA2.8 and pTR-CBA-hGAA3.1 with SnaBI-StuI, and replacing the CMV promoter and 5′ coding region of GAA in p43.2-hGAA2.8 with the CBA promoter and 5′ portion of GAA from pTR-CBA-hGAA3.1. The rAAV reporter plasmid, pTR-CBA-Luc, was constructed by replacing the IRES-GFP cassette in UF12 with the firefly luciferase cDNA from pGL3 (Promega, Madison, Wis.) using like HindIII-SalI sites.

To validate that enzymatically active protein was produced in the absence of the 3′ UTR, p43.2-hGAA2.8, p43.2-hGAA3.1, pTR-CBA-hGAA2.8, and pTR-CBA-hGAA3.1 were transfected into 70% confluent 6-well dishes of 293 cells using 5 μg of plasmid DNA purified using a QIAprep kit (Qiagen, Valencia, Calif.) and 10 μL Lipofectamine™ (GIBCO™ Invitrogen Corporation, Carlsbad, Calif.) per well according to manufacturer's recommendations. The cells were harvested after 48 hours of culture, and media and cellular extracts analyzed for the production of active GAA protein by enzyme assay. Results from triplicate transfections indicated that p43.2-hGAA2.8 and pTR-CBA-hGAA2.8 expressed enzymatically active protein even in the absence of the 3′ UTR. In fact, significantly higher activity was detected in the media and extract of cells transfected with hGAA2.8 constructs when compared to hGAA3.1 plasmids.

Highly purified rAAV serotype 2 vectors (rAAV2-CMV-hGAA, rAAV2-CBA-hGAA, and rAAV2-CBA-Luc) were generated using published methods (Zolotukhin et al., Gene Ther. 6:973-985, 1999). Producer cells were cotransfected with expression plasmids (p43.2-hGAA2.8, pTR-CBA-hGAA2.8, or pTR-CBA-Luc) and a rAAV2 helper/packaging plasmid, pDG (Xiao et al., J. Virol. 72:2224-2232, 1998). After 48 hours of culture, the cells were lysed and crude lysate was first purified on an iodixanol gradient. Resulting viral fractions were pulled and further purified on a heparin column. Pure virus was concentrated and analyzed by dot-blot to determine the particle titer and infectious center assay to quantify infectious titer. Similar techniques were used to produce rAAV serotype 1 vector, rAAV1-CMV-hGAA, but pXYZ1 was used as the helper/packaging plasmid and the heparin column purification step was eliminated (Rabinowitz et al., J. Virol. 76:791-801, 2002).

In utero viral delivery: On day 15 of gestation, pregnant females were anesthetized using 0.03 mL/gm total body weight of 20 mg/mL Avertin (tribromoethanol in tert-amyl alcohol diluted in PBS) administered intraperitoneal. A midline laparotomy was performed on each pregnant female with the abdominal wall being retracted to expose the peritoneal cavity. Each horn of the uterus was exposed individually onto a prewarmed saline-moistened sponge. The liver of each fetus was identified and correctly positioned using a dissecting microscope. Up to 10 μL of saline, beads, or virus was injected into each fetus. Trypan blue dye was added to the injection medium to ensure a direct injection was achieved. A preloaded Hamilton syringe bearing a 33 gauge needle with beveled end and side pore 20 (Hamilton Company, Reno, Nev.) was inserted through the uterine wall into the fetal liver or peritoneal cavity. After the injections, the first horn was returned to the abdominal cavity and an identical procedure was performed on the second uterine horn. After replacing the entire uterus into the abdominal cavity, 1.0 mL of prewarmed saline was added to the cavity to ensure the contents were moist. The abdominal muscle layer was sewn using 5-0 prolene and the skin layer was closed using 5-0 vicryl. Ampicilin (2.4 μL/gm body weight of 0.1 g/mL stock) and Buprenorphine (0.1 mg/kg) were administered after the surgery to control infection and pain. Mothers were monitored until they regained consciousness after which they were returned to the colony and permitted to proceed with the pregnancy. Newborn pups were kept with their mothers for 1 month before weaning.

Perfusion, necropsy and tissue analysis: After anesthetizing the animals, they were secured on a perfusion tray and opened along their midline. The chest was opened to expose the heart, being careful not to damage the diaphragm in the process. A 24 gauge catheter was placed in the left ventricle of the heart and a syringe connected to perfusion tubing was then attached to the catheter. Preloaded PBS at pH 7.4 was then circulated through the heart for 5 minutes at a rate of 2 mL/min. After perfusion began, the jugular vein in the right side of the neck was cut-to release the perfusion outflow. After perfusion, organs were successively removed from the animal using sterile surgical utensils, first beginning with skeletal muscle removed from lower extremities, then gonad, spleen, kidney, liver, diaphragm, lung, heart, tongue, and brain. The tissues were snap frozen in liquid nitrogen and stored at −80° C. in Nunc Cryo Tube™ vials (Nalge Nunc International, Rochester, N.Y.) to be later analyzed by activity assays, western analysis, and rAAV genome copy number. In particular, liver specimens from the florescent bead experiment were frozen in Tissue-Tek® O.C.T Compound embedding medium (Sakura Finetek, Inc., Torrance, Calif.) and hardened in an isopentane bath cooled by dry ice. Cryosections (10 μm) were cut, mounted, and photographed by fluorescence microscopy.

Tissues isolated for electron microscopy and histology were taken after first perfusing the mice with PBS for 5 minutes followed by 5 minutes of fixative (2% paraformaldehyde/1% glutaraldehyde in PBS, pH 7.4). Skeletal muscle, liver, diaphragm, and heart were removed, dissected into very small cubes, and stored overnight in 2% glutaraldehyde. They were rinsed in 0.1 M sodium cacodylate buffer and incubated at 4 C. in 2% osmium tetroxide in cacodylate buffer for 1 hour. They were then rinsed twice in cacodylate buffer, dehydrated in a series of graded alcohol solutions, rinsed in 100% propylene oxide, and embedded in TAAB resin (Marivac, Halifax, Canada). All other reagents were purchased from Electron Microsopy Sciences (Fort Washington, Pa.). Thick sections (1 μm) were stained with Schiff's reagent followed by toluidine blue and photographed using light microscopy. Thin sections (0.1 μm) were stained with lead citrate and uranyl acetate (Electron Microscopy Sciences, Fort Washington, Pa.), and photographed with a Zeiss EM10 transmission electron microscope at 80 kV.

Luciferase Expression Assay: The Luciferase Assay System (Promega, Madison, Wis.) was used to quantify the expression of luciferase. The samples were prepared by homogenization in 300 μL of water. Then 20 μL of the supernatant and 100 μL of luciferase assay substrate diluted in assay buffer were added to a glass test tube and incubated at room temperature for 20 minutes. The intensity of light emitted from the reaction was detected using the Monolight® 2010 luminometer (BD Biosciences, Mississauga, ON). Luciferase expression was reported as relative light units per μg protein as determined by DCProtein Assay (Bio-Rad, Hercules, Calif.).

Acid α-Glucosidase Activity Assay: GAA naturally cleaves the α1,4-bond of glycogen, and in this fluorimetric assay converts synthetic substrate 4-methylumbelliferyl-α-D-glucopyranoside (4-MUG, Calbiochem-Novabiochem Corp., San Diego, Calif.) to 4-methylumbelliferone (4-MU) and glucose. Snap frozen tissues were homogenized in water using a PowerGen 35 homogenizer (Fisher Scientific, Pittsburgh, Pa.) and cell pellets were resuspended in water and lysed by 3 freeze/thaw cycles. Lysates were isolated by centrifugation at 14000 rpm for 2 minutes. Then, 20 μL of tissue or cell extract was added to each well in triplicate to a black 96-well Costar® plate (Corning, Inc., Acton, Mass.). Next, 40 μL of substrate solution (2.2 mM 4-MUG in 0.2 M sodium acetate pH 3.6) was added to each well. The plate was covered with parafilm and incubated at 37° C. for 1 hour. Then each reaction was stopped by adding 200 μL of 0.5 M sodium carbonate (pH 10.7). Standards ranging from 1 to 500 μM 4-MU were included on each plate. Concentrations of 0, 3.125, 6.25, 12.5, 25, 50, 100, and 500 μM 4-MU in a volume of 20 μL were added per well in addition to 40 μL of 0.2 M sodium acetate, pH 3.6 and 200 μL of 0.5 M sodium carbonate (pH 10.7). Fluorescence was then measured using an FL×800 Microplate Fluorescence Reader (Biotek Instruments, Inc., Winooski, Vt.) by exciting the sample at 360 nm and detecting at 460 nm. Acid α-glucosidase specific activity was quantified in nmoles of substrate hydrolyzed (nmoles 4-MUG/hr/mg protein) based on a standard curve of 4-MU concentrations and standardized by protein concentration determination by DC Protein Assay (Bio-Rad, Hercules, Calif.).

Protein Concentration Determination: The DC Protein Assay kit (Bio-Rad, Hercules, Calif.) was used according to manufacturer's suggestions to determine protein concentration of tissue homogenates. The colorimetric assay is based on the Lowry method of protein determination. Dilutions of bovine serum albumin ranging from 0.2 to 1.5 μg/μL were used to create a standard curve. Standards and samples (5 μL) were added to a clear 96-well microtiter plate, followed by the addition of reagent A (25 μL) and regeant B (200 μL). The reaction was allowed to proceed for 15 minutes at room temperature after which absorbance at 750 nm was determined using a μQuant microplate reader (Biotek Instruments, Inc., Winooski, Vt.). Protein concentrations were reported as μg/μL of sample.

Acid α-Glucosidase Staining of Tissues: GAA expression in tissues was visualized using a method similar to that used to detect β-galactosidase. Acid α-glucosidase was detected by cytochemical staining using the synthetic substrate 5-bromo-4-chloro-3-indolyl-α-D-glucopyranoside (X-Gluc, Calbiochem-Novabiochem Corp., San Diego, Calif.). After the animals were perfused with PBS and the tissues harvested, a portion was placed in 4% paraformaldehyde for 1 hour. After washing with PBS, X-Gluc stain (0.25 mM potassium ferricyanide, 0.25 mM potassium ferrocyanide, 1 mM magnesium chloride, 1 mg/mL X-Gluc in PBS reduced to pH 3.6) was added and the samples were incubated at room temperature overnight. The tissues were photographed using a digital camera attached to a dissecting microscope.

Western blotting: Rabbit polyclonal antiserum was raised against placentally derived human GAA as previously described (Pauly et al., Gene Ther. 5:473-480, 1998). The antiserum was used for western blotting to detect hGAA protein. A total of 5 μg of protein from tissue homogenates was applied to Novex® 8% Tris-Glycine gels (Invitrogen Life Technologies, Carlsbad, Calif.) and separated at 125 V for approximately 2 hours. After transfer to nitrocellulose filters, blots were probed with 1:1000 dilution of primary antibody followed by 1:5000 dilution of peroxidase-labeled anti-rabbit IgG (Amersham Biosciences Corp., Piscataway, N.J.) and detected using the ECL+Plus chemiluminescence kit (Amersham Biosciences Corp., Piscataway, N.J.). Human placental GAA was included on each blot as a positive control.

Quantification of genome copies by Quantitative-Competitive PCR (QC-PCR): Competitor plasmid construct, p43.2-hGAA2.8-5′ del, was created by digestion of p43.2-hGAA2.8 with KpnI-SacII followed by T4 polymerase extension of 5′ overhangs and blunt-end ligation. Approximately 350 nucleotides from-the 5′ end of the GAA gene were removed. Primers were designed to amplify 595 nt of rAAV-CMV-hGAA genomic DNA and 239 bp of the p43.2-hGAA2.8-5′ del competitor template. The 5′ primer was located in the multiple cloning site after the CMV promoter of p43.2 and 3′ primer was positioned beginning at nucleotide 514 of the hGAA coding sequence.

Total DNA was isolated from snap-frozen specimens using DNeasy® tissue kit (Qiagen, Valencia, Calif.). An RNase digestion step was included to remove any mRNA species which may contaminate the QC-PCR. Reactions were arranged by adding 200 ng of total DNA, competitor plasmid DNA (ranging from 0 to 10⁸ copies), 20 pmol of each primer, and water to Ready-To-Go™ PCR beads (Amersham Biosciences Corp., Piscataway, N.J.). The reaction contained 1.5 mM MgCl in a total volume of 25 μL according to manufacturer's suggestions. Samples were subjected to 30 cycles of denaturation at 95° C. for 30 sec, annealing at 60° C. for 30 sec, and elongation at 72° C. for 30 sec using a RoboCycler® Gradient 96 thermocycler (Stratagene, La Jolla, Calif.). QC-PCR samples were separated on a 2% agarose gel and photographed using the Eagle Eye™ II imaging system (Stratagene, La Jolla, Calif.). The amplified products were quantified using Imagequant™ software (Amersham Biosciences, Piscataway, N.J.). Intensities of products from amplified genomic rAAV-CMV-hGAA and competitor plasmid DNA were plotted on the same graph using SigmaPlot 2001 software (SPSS, Inc., Chicago, Ill.). The point where both lines crossed was considered the point of equal amplification. Given that the amount of competitor and sample template is equal at this point, the number of vector genome copies present in the sample was approximated. Data were reported as vector genome copies/diploid cell after converting from vector genome copies/200 ng DNA using a conversion factor of 5 pg DNA/diploid nucleus.

In vitro assessment of diaphragm contractile function—Diaphragm muscle strip preparation: Mice were anesthetized via intraperitoneal injection of sodium pentobarbital (65 mg/kg). After reaching a surgical plane of anesthesia, the diaphragm was surgically excised, with the ribs and central tendon attached, and placed in a cooled dissecting chamber containing Krebs-Henseleit solution equilibrated with a 95% O₂/5% CO₂ gas mixture. A single muscle strip (3-4 mm width) was cut from the ventral-costal diaphragm parallel to the connective tissue fibers.

Segments of the rib and central tendon were used to facilitate attachment of the strip to two lightweight Plexiglas clamps. Using these clamps, the muscle strip was suspended vertically in a water-jacketed tissue bath (Radnoti, Monrovia, Calif.) containing Krebs-Henseleit solution equilibrated with a 95% O₂/5% CO₂ gas mixture. The bath was maintained at 37±0.5° C., pH˜7.4±0.05, and osmolality ˜290 mOsmol. In order to assess isometric contractile properties, the clamp attached to the central tendon was connected to a force transducer (Model FT03, Grass Instruments, West Warwick, R.I.). Transducer outputs were amplified and differentiated by operational amplifiers and underwent A/D conversion using a computer-based data acquisition system (Polyview, Grass Instruments).

Determination of optimal length-tension relationship (Lₒ) and isometric force-frequency relationship (FFR): After a 15-minute equilibration period, in vitro contractile measurements began with empirical determination of the muscle strip's optimal length (Lₒ) for isometric-titanic tension development. The muscle was field-stimulated (Model S48, Grass Instruments) along its entire length using custom-made platinum wire electrodes. Single twitch contractions were evoked, followed by step-wise increases in muscle length, until maximal isometric twitch tension was obtained. Once the highest twitch force was achieved, all contractile properties were measured isometrically at Lₒ. Peak isometric titanic force was measured at each of the following frequencies: 10, 20, 40, 80, 100, 150, and 200 Hz. Single 500 ms trains were used, with a four-minute recovery period between trains to prevent fatigue. At the conclusion of each study, calipers were used to measure Lₒ before the strips were removed from the apparatus.

Measurement of the diaphragm strip cross-sectional area: After removing the muscle strips from the Plexiglas clamps, the muscle tissue was carefully dissected from the rib and central tendon, blotted dry, and weighed. The muscle cross-sectional area (CSA) was determined as using the equation CSA (cm²)=[muscle strip mass (g)/fiber length Lₒ (cm)×1.056 (g/cm³)], where 1.056 g/cm³ was the assumed density of muscle. The calculated CSA was used to normalize isometric tension, which is expressed as N/cm².

Results

Analysis at the cellular level using electron microscopy revealed abnormal glycogen deposition within various tissues of Gaa⁻/⁻ mice and how this could result in abnormal muscle function. Heart, skeletal muscle, diaphragm, and liver of 1-month-old Gaa⁻/⁻ and normal (C57B6/129-SvJ) mice were examined by electron microscopy. Even at this early age, enormous glycogen inclusions were seen crowding muscle fibers of Gaa knockout heart, skeletal muscle, and diaphragm. Massive amounts of glycogen were also identified in knockout liver. Deposits of glycogen were rarely observed among normal tissues. Some glycogen was found associated with lysosome-like membrane structures, while in other cases, deposits were seen grouped in the cytoplasm without defined membrane structures. Most aggregates of glycogen observed in Gaa⁻/⁻ skeletal muscle were associated with what appeared to be cellular debris. Glycogen seemed to take on different forms among knockout tissues. For instance, in the heart, the glycogen seemed dense, while it was more dispersed in skeletal muscle and liver. This could be an artifact caused by fixation differences among tissues, with dense glycogen indicating better preservation of the tissue.

Localization of fluorescent beads to the liver after in utero hepatic injection: In utero hepatic delivery of rAAV was focused on with the anticipation of achieving high level gene expression of GAA in the liver. GAA produced in the liver could be secreted and dispersed via the circulation to target tissues such as heart, diaphragm, and skeletal muscle. In the target tissue, the protein would be escorted to the lysosome by mannose 6-phosphate receptor-mediated endocytosis. Localization of the injected medium after in utero hepatic injections was investigated using 10 μL of 0.1% w/v 30 nm fluorescent beads. The beads were introduced by injecting through the uterine wall and into the red-pigmented liver of a 15 p.c. CD-1 fetus. Fluorescent beads were found localized in the liver at the site of injection. This is an important aspect since the diameter of the fluorescent beads (30 nm) and rAAV (approximately 25 nm) are similar. These results showed that rAAV can be delivered to the liver of the developing murine fetus and that the fetus could be carried to term.

Survival study of Gaa⁻/⁻ in utero injections: A total of 294 Gaa⁻/⁻ fetuses were injected at 15 days gestation from 50 timed-pregnant females, of which 167 fetuses were brought to term leading to a surgery survival rate of 60.5%, compared to 100% normal birth rate. Of the 148 injected mice allowed to reach a weaning age of 3 weeks, 108 remained. This indicated a post-birth survival rate of 73.0%, a rate similar to animals of this strain not in utero-treated. Most of these deaths were due to maternal neglect or cannibalization which is normally seen among this and other knockout strains.

High level transduction of diaphragm muscle through in utero delivery of rAAV serotype 2 to the liver and peritoneal cavity: The level of luciferase expression was determined in several tissue types 1 month after in utero hepatic delivery of 3×10⁷ infectious particles of rAAV2-CBA-Luc to Gaa⁻/⁻ fetuses on day 15 of gestation. Animals were sacrificed at 1 month and assayed for luciferase expression. Expression levels were highest in the diaphragm and liver, while no significant expression was detected in kidney, spleen, skeletal muscle, gonad, lung, heart, brain, and tongue of 1-month-old Gaa⁻/⁻ vector-treated mice. Luciferase expression levels of individual samples were measured and activity values of saline were averaged in rAAV2-CBA-Luc-treated tissues. More than 100-fold higher luciferase expression was detected in rAAV in utero-treated diaphragms compared with saline-treated mice. It is likely that high-level diaphragmatic transduction occurred through intraperitoneal exposure to the rAAV2 vector.

In utero transduction of diaphragm muscle leads to production of normal levels of enzymatically active GAA protein in Gaa⁻/⁻ mice: Gaa⁻/⁻ fetuses were injected at 15 days gestation with 2×10⁸ infectious particles of rAAV2-CBA-hGAA, 1×10⁹ infectious particles of rAAV2-CMV-hGAA, 3×10⁷ infectious particles of rAAV2-CBA-Luc, or saline. Four C57B6/129-SvJ normal mice, four saline and rAAV2-CBA-Luc-treated Gaa⁻/⁻ negative controls, eight rAAV2-CBA-hGAA (numbered as animals 1-8) and four rAAV2-CMV-hGAA (1-4) treated Gaa⁻/⁻ mice were sacrificed at 1 month of age to isolate liver, kidney, spleen, skeletal muscle, gonad, diaphragm, lung, heart, brain, and tongue for GAA activity assays. Again, vector-treated diaphragms yielded the highest enzyme activity, while levels in the other tissues tested did not reach significance. Individual enzyme values and averaged values within experimental groups were measured. Average GAA enzyme activity in normal diaphragm was 23.6 nmol 4-MUG/hr/mg protein, and this level was reached in animals rAAV2-CBA-hGAA-2 (26.2 nmol 4-MUG/hr/mg protein) and rAAV2-CMV-hGAA-1 (27.3 nmol 4-MUG/hr/mg protein) while higher than normal levels were observed in rAAV2-CMV-hGAA-3 and -4 (44.5 and 40.0 nmol 4-MUG/hr/mg protein). On average, the rAAV2-CBA-hGAA-treated group reached almost 25% of normal GAA activity, while the rAAV2-CMV-hGAA group surpassed normal levels. The rAAV2-CMV-hGAA group attained higher levels than the rAAV-CBA-hGAA group possibly because the CMV-treated group received five times more vector, although differences in promoter strength can not be excluded.

To determine which isoform of hGAA protein was being detected enzymatically, western analysis of diaphragm extract from the same 1-month-old rAAV2-CBA-hGAA and rAAV2-CMV-hGAA in utero-treated animals, as well as Gaa⁻/⁻ untreated and normal diaphragm, was performed using a polyclonal antibody specific for hGAA. GAA purified from human placenta was used as a control to show the predominant isoforms, 95-kD precursor and 76-and 67-kD processed forms. An unknown cross-reacting protein (about 50 kD) was detected in all samples, but served as a loading control. Endogenous murine GAA in the normal diaphragm extracts was not detected by this antibody, which is specific against hGAA. As expected, no signal was detected from untreated Gaa⁻/⁻ diaphragm. Those animals expressing detectable levels of hGAA by enzyme assay, rAAV2-CMV-hGAA-1 and -2 and rAAV2-CBA-hGAA-1, -3, and -4, revealed the presence of the catalytically active 76-kD processed form. Protein levels detected by western analysis were consistent with the relative levels of measured enzymatic activity.

Higher level expression achieved using rAAV serotype 1: Based on previous experimentation, it was discovered that rAAV serotype 1 is superior over serotype 2 in transducing muscle tissue when delivering the human GAA cDNA to the Gaa⁻/⁻ mouse (Fraites, Molec. Ther. 5:1-8, 2002). To determine if in utero delivery of rAAV serotype 1 vector would provide superior tropism and level of transduction compared to that previously found using serotype 2, 8.14×10¹⁰ particles of rAAV1-CMV-hGAA were delivered to Gaa⁻/⁻ fetuses at 15 days gestation. After allowing the vector-treated pups to reach 1 month of age, they were sacrificed to isolate liver, kidney, spleen, skeletal muscle, gonad, diaphragm, lung, heart, brain, and tongue for GAA activity assays. Once again, GAA activity was detected only in diaphragm. No other tissues tested expressed a significant level of GAA. In several cases, diaphragmatic transduction with rAAV serotype 1 resulted in almost 10-fold higher GAA activity, surpassing both normal controls as well as rAAV serotype 2 in utero-treated Gaa⁻/⁻ animals.

GAA expression in the diaphragm was examined by cytochemical staining using 5-bromo-4-chloro-3-indolyl-α-D-glucopyranoside (X-Gluc), a synthetic substrate similar to X-Gal used to detect β-galactosidase. This staining procedure identified cells expressing GAA activity higher than normal levels, since no blue cells were observed after X-Gluc staining of normal C57B6/129-SvJ diaphragms. After fixation, half of each of the in utero-treated diaphragm was immersed into the X-Gluc solution overnight and photographed to document the level of GAA expression. Some diaphragms showed significant blue staining while others were indistinguishable from the untreated Gaa⁻/⁻ control.

The level of GAA activity determined from the other half of the diaphragm indicated that the amount of staining was relative to the level of activity. The diaphragms with the highest intensity of staining reached over 100 nmol 4-MUG/hr/mg protein (rAAV1-CMV-hGAA-2, -6, and -8) with one yielding 824 nmol 4-MUG/hr/mg protein (rAAV1-CMV-hGAA-2); those with intermediate staining attained normal levels of approximately 20 nmol 4-MUG/hr/mg protein; and those that lacked staining had no detectable GAA activity (rAAV1-CMV-hGAA-3, -4, and -7).

By western analysis, it was discovered that the 76-kD mature form of GAA was responsible for the observed activity. The intensity of the 76-kD band observed in each of the in utero-treated diaphragms was consistent with what was determined by activity assay and X-Gluc staining with the exception of rAAV1-CMV-hGAA-5. Although an intermediate level of X-Gluc staining was observed, GAA activity analysis revealed only 18 nmol 4-MUG/hr/mg protein of active protein was present. Correlating with the activity assay, western analysis indicated a very low level of mature enzyme was present. However, there was a predominant band of a molecular weight higher than the 95-kD intermediate form visible in the placental control lane. It was likely to be the 110-kD precursor form of the protein. This partially explains why the X-gluc staining of this diaphragm did not correlate with the activity assay. The higher molecular weight species detected by western analysis may be able to enzymatically cleave the X-Gluc substrate more efficiently than 4-MUG, which was used in this activity assay. The 110-kD precursor form of GAA exhibits low level activity on particular substrates, but this activity increases as the protein is further processed.

Prevention of lysosomal glycogen accumulation in GAA⁻/⁻ mice in utero treated with rAAV2-hGAA: To determine if lysosomal glycogen accumulation associated with GSDII and observed in this animal model (rAAV-hGAA in utero-treated Gaa⁻/⁻ animals were exposed to vector-produced hGAA enzyme at an early stage in development) was prevented in the treated animals, PAS was used to stain intracellular glycogen deposits of normal C57B6/129-SvJ, untreated Gaa⁻/⁻, and rAAV1-CMV-hGAA-treated Gaa⁻/⁻ diaphragm sections from 1-month-old mice. At this early stage in development, glycogen inclusions were evident in the diaphragm of untreated Gaa⁻/⁻ mice. Numerous pink-stained glycogen-filled lysosomes scattered the field of the untreated Gaa⁻/⁻ diaphragm. Lysosomes swollen with undegraded glycogen were found both at the cell periphery and among the fibers of the microtubes. Conversely, all of the myofibers of the normal and vector-treated Gaa⁻/⁻ diaphragms were free of stain, making it impossible to differentiate between the two. These findings were confirmed by electron microscopy analysis. Extremely large lysosomes full of glycogen were present among the muscle fibers of untreated Gaa⁻/⁻ diaphragm but were not seen in normal tissue or treated samples expressing normal levels of GAA. However, transducing the diaphragm to act as a factory for producing secreted GAA to treat other tissues was not successful. For instance, heart tissue from one animal had significant PAS positive material. Control heart tissue from C57B6/129-SvJ and untreated Gaa⁻/⁻ mice was also included.

Level of GAA expression in diaphragm following in utero delivery of rAAV1-CMV-hGAA depends on vector genome copy number: Quantitative-competitive PCR was performed to determine vector genome copy number among 1-month-old Gaa⁻/⁻ mice after in utero delivery of rAAV1-CMV-hGAA. The tissues assayed were from the same diaphragms previously described above. Total DNA was extracted from treated and untreated diaphragms and 200 ng of DNA was mixed with increasing copies of plasmid competitor DNA. PCR was performed using a pair of primers that detected both the rAAV1-CMV-hGAA vector (595 bp product) and the CMV-hGAA3′ deleted plasmid competitor (239 bp product) at equal efficiency.

The 595 bp rAAV1-CMV-hGAA amplified product was detected in each treated diaphragm sample, rAAV1-CMV-hGAA-1 through -8, but to varying levels. All samples indicated the presence of vector genomes whether or not GAA protein was detected by staining, enzyme assay, or western analysis. The 595 rAAV1-CMV-hGAA amplified product was not detected in untreated Gaa⁻/⁻ animals. PCR was performed after mixing increasing amounts of competitor DNA (10⁰ to 10⁸ copies) and 200 ng of total DNA isolated from diaphragms of Gaa⁻/⁻ untreated mice or Gaa⁻/⁻ mice in utero-treated with 8.14×10¹⁰ particles of rAAV1-CMV-hGAA . Densitometry was performed to more accurately determine vector genome copy number present within 200 ng of diaphragm DNA and this value was converted into vector copies/diploid genome based on a 5 pg total DNA/cell conversion factor. Control reactions were completed in which β-actin was amplified from 200 ng DNA from each sample. This showed that the amount of DNA added to each QC-PCR reaction was relatively the same.

The general trend indicated by the QC-PCR experiments was that as the copy number of vector genomes per diploid cell increased, the resulting GAA activity also increased. Diaphragm rAAV1-CMV-hGAA-2, which had the highest level of GAA activity (824 nmol 4-MUG/hr/mg protein), contained 50 estimated vector copies per diploid genome. Diaphragm rAAV1-CMV-hGAA-1, which had a normal level of GAA activity (24 nmol 4-MUG/hr/mg protein) and over 10-fold less activity than rAAV1-CMV-hGAA-2, contained only a slightly lower copy number at 20 estimated vector copies per diploid genome. Even sample rAAV1-CMV-hGAA-7, which had minimal GAA activity (1.2 nmol 4-MUG/hr/mg protein), was found to contain significant vector genomes with 2.5 estimated vector copies per diploid genome detected. Even though significant levels of rAAV1-CMV-hGAA vector genome copies were present in all treated diaphragms, there was an optimal threshold of genome copies which must be present in order to produce a detectable level of GAA.

QC-PCR was also performed on the four livers from treated mice in which the diaphragms exhibited high GAA activity (rAAV1-CMV-hGAA-2, -5, -6, and -8). Three of those livers tested had positive amplification signals, and were found to contain on average, 0.1 estimated vector copies per diploid genome. Even though it was uncertain whether the livers sampled in this experiment were actually the lobes directly injected, this indicated that there were vector genomes present in most the livers tested. For the most accurate representation of vector genome copies in the liver, QC-PCR should be performed on DNA representative of the entire liver.

Diaphragmatic transduction following in utero delivery of rAAV1-CMV-hGAA results from intraperitoneal exposure to the vector: To determine if intraperitoneal exposure of rAAV-hGAA after hepatic in utero injections was the source of diaphragmatic transduction, several intraperitoneal in utero injections were performed. 8.14×10¹⁰ particles rAAV1-CMV-hGAA were delivered to 15 p.c. Gaa⁻/⁻ fetuses via the intraperitoneal cavity and diaphragms harvested from three animals at 1 month of age. The tissues were assayed by X-Gluc staining, GAA activity, western analysis, and QC-PCR. Each diaphragm was positive to varying extent for X-Gluc staining, GAA activity, and vector genomes (Table 1).

At least normal levels of GAA activity were achieved in all treated samples (Table 1). Western analysis confirmed the presence of the 76-kD mature form of GAA. QC-PCR was used to analyze rAAV1-CMV-hGAA-1 through -3 diaphragms for vector genome copy number (Table 1). Every IP in utero-treated diaphragm was positive for vector genomes resulting in 1 to 100 estimated vector copies per diploid genome. There were some discrepancies between relative level of GAA activity and vector genome copy number among these samples. For instance, rAAV1-CMV-hGAA-2 exhibited significantly higher GAA activity than rAAV1-CMV-hGAA-3, but rAAV1-CMV-hGAA-3 contained several more vector copies per diploid genome. This could be due to the unequal transduction over the entire diaphragm muscle. Protein for GAA activity assays and western analysis was isolated from a different part of the diaphragm than what was used to isolate DNA for QC-PCR. Nevertheless, all in utero IP-treated Gaa⁻/⁻ diaphragms resulted in higher than normal levels of GAA activity and all were positive for rAAV1-CMV-hGAA vector genomes.

TABLE 1
Biochemical and genomic analysis of diaphragms after
IP in utero delivery of rAAV1-CMV = hGAA.

| | GAA Activity | QU-PCR (estimated |
|---|---|---|
| Intraperitoneal in utero | assay (nmol | vector genome |
| delivery rAAV1-CMV- | 4-MUG/hr/mg | copies/diploid |
| hGAA2.8 | protein) | genome) |
| Gaa⁻/⁻(n = 4) | 0 | 0 |
| WT C57B6/129-SvJ (n = 4) | 23.6 | N/A |
| rAAV1-CMV-hGAA #1 | 32.6 | 1 |
| rAAV1-CMV-hGAA #2 | 559.0 | 15 |
| rAAV1-CMV-hGAA #3 | 154.1 | 100 |

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising a step of administering to a cell an rAAV virion comprising: (a) an acid alpha-glucosidase polypeptide-encoding polynucleotide interposed between a first AAV inverted terminal repeat and second AAV inverted terminal repeat; and (b) an AAV serotype 1 capsid protein.

2. The method of claim 1, wherein the acid alpha-glucosidase polypeptide is a human acid alpha-glucosidase polypeptide.

3. The method of claim 1, wherein the acid alpha-glucosidase polypeptide-encoding polynucleotide is operably linked to an expression control sequence.

4. The method of claim 3, wherein the expression control sequence is a promoter.

5. The method of claim 4, wherein the promoter is a CMV immediate early promoter.

6. The method of claim 1, wherein the cell is a mammalian cell.

7. The method of claim 6, wherein the mammalian cell is a muscle cell.

8. The method of claim 7, wherein the muscle cell is derived from an animal having lower than wild-type acid alpha-glucosidase polypeptide levels.

9. The method of claim 6, wherein the cell is located within a mammalian subject.

10. The method of claim 9, wherein the subject is a post-natal animal.

11. The method of claim 9, wherein the subject is a fetus.

12. The method of claim 9, wherein the step of administering the rAAV virion is performed by parenteral administration into the subject.

13. The method of claim 12, wherein the parenteral administration is injection.

14. The method of claim 13, wherein the injection is IM injection.

15. The method of claim 13, wherein the injection is into a blood vessel.

16. The method of claim 9, wherein the mammalian subject has lower than wild-type acid alpha-glucosidase polypeptide levels.

17. The method of claim 16, wherein the step of administering the rAAV virion results in increased acid alpha-glucosidase polypeptide levels in the mammalian subject.

18. The method of claim 17, wherein the resulting acid alpha-glucosidase polypeptide levels are at least at wild-type levels.

19. The method of claim 17, wherein the resulting acid alpha-glucosidase polypeptide levels are at greater than wild-type levels.

20. The method of claim 9, wherein the mammalian subject exhibits clinical symptoms associated with low alpha-glucosidase polypeptide levels, and wherein the symptoms are ameliorated after the step of administering the rAAV virion.
